(19) United States
(12) Patent Application Publication
(10) Pub. No.: US 2026/0215812 A1
(43) Pub. Date: Jul. 30, 2026

(54) A Perineum Damage-Reducing Device and a Method for Reducing Damage During Vaginal Child Delivery

(71) Applicant: Oasicare ApS, København V (DK)

(72) Inventors: Ditte Marie Fog Ibsen, København V (DK); Julia Sand, København V (DK)

(21) Appl. No.: 19/146,912

(22) PCT Filed: Jan. 5, 2023
(86) PCT No.: PCT/DK2023/050003

§ 371 Date: Jul. 9, 2025

(51) Int. Cl. A61B 17/42

(52) U.S. Cl. CPC A61B 17/42

(57) ABSTRACT

A perineum damage-reducing device for reducing damage during vaginal child delivery includes a first part configured to be inserted into a female body via introitus vagina at a posterior part of introitus vagina, and a second part configured to extend outside the female body away from the posterior part of introitus vagina towards anus. The device comprises a sheet of flexible material, and at least one reinforcing element, which stabilizes said sheet and extends over both said first and said second part. The second part further comprises at least one attachment element at the inner surface configured for attachment to a surface of the female body. In one embodiment the first and second parts meet at a bend and extend at an angle in relation to each other in a state of delivery.

### CROSS-REFERENCE TO RELATED APPLICATIONS

This application is the national phase of, and claims priority to, International Application No. PCT/DK2023/050003, filed 5 Jan. 2023.

### FIELD OF THE INVENTION

The invention relates to perineum damage-reducing devices for reducing damage during vaginal child delivery, comprising: a first part configured to be inserted into a female body via introitus vagina at a posterior part of introitus vagina; and a second part configured to extend outside the female body away from the posterior part of introitus vagina towards anus; an inner surface extending over the first part and the second part and being configured to be in contact with surfaces of the female body; an outer surface opposite to said inner surface; said inner surface and said outer surface being formed by a sheet of flexible material; and said perineum damage-reducing device further comprising at least one reinforcing elements for stabilizing said sheet. The invention further relates to a method for reducing damage during vaginal child delivery.

### BACKGROUND OF THE INVENTION

Damage to the perineum, i.e. the area between the opening of the vagina and the anus, is a common complication following vaginal delivery and can cause long-term discomfort. Damage to the perineum may come in the form of perineal tears, which are lacerations of the skin and other soft tissue structures which, in women, separate the vagina from the anus. Ruptures often start at the posterior part of the introitus vagina, i.e. the area of the introitus vagina closest to the anus and to the back of the body. While all ruptures give discomfort, more severe ruptures involving the anal sphincters or urethral sphincters may cause incontinence.

Up to 9 in 10 first-time mothers who have a vaginal child delivery will have some sort of perineum damage, making it the most common form of obstetric injury. Even though a sphincter injury is detected and sutured immediately after delivery, up to 50% of patients experience faecal or urinary incontinence, pain, or sexual dysfunction at some point. These complications can have important consequences for the woman's quality of life.

Perineal protection during vaginal child delivery can be achieved manually by healthcare personnel, usually midwives, where they place their thumb and index fingers alongside the introitus vagina, also referred to as the vaginal opening, to support the perineum. However, many of them have not received proper training, especially not in developing countries.

It has therefore been attempted to provide devices as described above to reduce the number of tears and protect the perineum during vaginal child delivery. Such a device is known from WO 09101186 A1. This device is specifically adapted for protecting the tissue in the posterior part of the introitus vagina and works very well, but it too requires the involvement of trained healthcare personnel.

### SUMMARY OF THE INVENTION

It is therefore an object of the invention to provide an alternative device that can be handled by unskilled birth helpers.

According to an aspect of the invention, this and further objects are achieved with a device as described above further comprising wherein the at least one reinforcing element extends over both said first and said second parts, and in that the second part comprises at least one attachment element at the inner surface configured for attachment to a surface of the female body.

The combination of the attachment element and the reinforcing element extending over both the first and the second part facilitates handling of the perineum damage-reducing device, as it ensures that the device is positioned correctly and remains in place during use. Only the second part needs to be attached to the female body. The reinforcing element extending into the first part ensures that the first part will be positioned along the vaginal wall at the posterior part of the introitus vagina as a consequence of the positioning of the second part. This means that the unskilled birth helpers only needs to ensure that the second part is attached properly. This is especially advantageous as it may be difficult to see the first part once inserted, and thus difficult to know if it is positioned correctly. This entails that the device can be applied by unskilled birth helpers, such as birth helpers in developing countries, who have often received no or only very basic training, and who therefore may be dependent on application instructions in the form of, for instance, illustrations or an app. While the device may be especially helpful for unskilled birth helpers, it may also be useful for trained birth helpers, such as health workers, midwives, and doctors.

The reinforcing element extending over both the first part and the second part may also result in that the first part becomes repositioned as a result of a change of shape of the second part. During vaginal child delivery, introitus vagina expands causing the adjacent areas to expand as well, and consequently the second part may expand or otherwise change shape. This means that the perineum damage-reducing device may respond to changes in the shape of the female body during child delivery so that the first part remains in contact with the vaginal wall even though not attached to it.

The at least one attachment element further ensures that the device will remain attached to the female body during the entire use.

An additional advantage of the invention is that the perineum damage-reducing device provides a protective effect via dispersion of the perineal tension over a wide surface area, not only over the area to which the second part is attached but also to the vaginal wall via the first part. Moreover, the device may slow down the expansion time of the perineum, thus minimizing the risk of ruptures.

Providing at least one reinforcing element that extends over both the first part and the second part, also allows the device to be easy to handle and position for an unskilled birth helper. The reinforcing element extending over both parts provides stability and prevents the device from sticking together or getting twisted.

In a preferred embodiment, the sheet of flexible material is elastic. Elastic refers to the elasticity of a material, which is the tendency of solid materials to return to their original shape after forces are applied on them. This is an advantage when the introitus vagina expands and causes the adjacent surfaces to expand as well. When expanded, the sheet will thereby seek to return to its original shape and may thereby resist expansion of the perineum.

In one embodiment, the flexible sheet may be able to expand to double or triple its size.

In one embodiment, the inner surface of said first part comprises a smooth surface. A smooth surface comprises a non-adhering surface. The smooth surface may facilitate easy handling of the device, as the first part will not stick to undesired places. This is particularly useful in cases where the first part is initially placed incorrectly, which may happen frequently especially for unskilled birth helpers, as the first part may not be visible due to its position inside the vagina. When the second part has been attached to the surfaces of the female body, the first part will consequently be free to reposition itself and fall into place as described above.

In one embodiment, only the second part comprises an attachment element.

In one embodiment, the second part comprises two side sections and a middle section, and the at least one reinforcing element extends through the middle section and outwardly towards edges of said side sections. By providing reinforcing elements that extend over a big area of the device, the device becomes stable and easy to handle. This also ensures a good force transmission from the second part to the first part as there is a large contact area between the reinforcing element and the sheet. A large reinforcing element also allows for a good transmission of forces between the device and the female body.

The at least one reinforcing element may comprise two sections extending from the first part to the second part, said sections being interconnected at the first part. The reinforcing element may extend to both side sections.

It is also possible to provide two or more separate reinforcing elements, each extending over both said first and said second part. In that case one reinforcing element may extend into one of the side sections, while another reinforcing element extends into the other side section.

In one embodiment, the at least one reinforcing element further extends inwardly back towards the middle section of the second part. The section of the reinforcing element extending in the second part may then have a rounded shape, preferably in a U-shape. This may further stabilize the device as the contact area between the reinforcing element and sheet is relatively big.

The reinforcing element is preferably without sharp edges to avoid damage to the sheet.

In one embodiment, said first part and said second part meet at a bend and extend at an angle in relation to each other, said angle preferably being in the range of 20-70 degrees, more preferably 30-60 degrees, more preferably 30-40 degrees. These angles suit the anatomy of the average female particularly well, which has proven to be useful, as the angles help facilitate correct attachment of the device and that the device remains in place during use.

In one embodiment, in a state of delivery, the device is pre-formed with the bend between the first and second parts. State of delivery refers to the state of the device at the time the user comes in possession of the device and includes the state of the device when applying the device to the body. Pre-formed indicates that the device is formed in a ready-to-use condition at the state of delivery. The ready-to-use condition further facilitates handling of the device as the device may already fit the anatomy of the female body and may not need further bending.

The pre-forming does not limit the device to one shape. The pre-formed device may still be adjusted.

In an embodiment, the sheet of flexible material has a thickening at the bend. The thickening at the bend is intended for maintaining the intended angle between the first and second parts, thus preventing the device from collapsing. The thickening, however, should allow some repositioning of the first and second parts relative to each other during use, for example as a consequence of the child pressing on the first part.

In one embodiment, the at least one reinforcing element is elastic, pulling the device towards the state of delivery. This may contribute to the ability of the device to follow changes in the shape of the female body during child delivery, such as the introitus vagina expanding during passing of the child. Elasticity of the reinforcing element also reduces the risk of breakage, which might result in the formation of sharp edges.

The reinforcing element may comprise different materials or it may comprise the same material as the sheet. In the case of the reinforcing element comprising the same material as the sheet, the material of the reinforcing element may have a higher density or compactness.

The reinforcing elements may comprise a polymeric material, such as elastomers, thermoplastic elastomers, mediprene thermoplastic elastomers, nylon, polypropylene, polyethylene, polyurethane, polycarbonate, vinyl, polytetrafluoreten, silicon, or textiles.

In an embodiment, the reinforcing elements may comprise a non-polymeric material, such as a metallic material.

In one embodiment, the reinforcing element has a higher stiffness than the sheet. This enables the reinforcing element to reinforce the device and thereby facilitate handling of the device. The stiffness refers to the extent to which an object resists deformation in response to an applied force. The stiffness ensures a stable reinforcing element.

In one embodiment, the reinforcing element is embedded in the sheet of flexible material between said inner surface and said outer surface. This may further facilitate handling of the device and may reduce the risk of any unintentional damage, caused by the reinforcing element, to the mother, the child, or the unskilled birth helper. For the same reason, the reinforcing element is preferably without sharp edges.

If the reinforcing element is not covered by the sheet element, it should preferably be biocompatible.

In a preferred embodiment, the sheet of flexible material comprises a polymeric material, such as elastomers, thermoplastic elastomers, mediprene thermoplastic elastomers, nylon, polypropylene, polyethylene, polyurethane, polycarbonate, vinyl, polytetrafluoreten, silicon, or textiles.

In one embodiment, the sheet of flexible material may comprise a non-polymeric material.

Flexible materials refer to materials having the ability to bend or compress easily without cracking under normal conditions. Flexible materials enable the device to fit different shapes which is advantageous in a dynamic environment, such as during a vaginal child delivery.

The sheet of flexible material may comprise one or more layers of the same material or different materials.

In one embodiment, the sheet of flexible material comprises a transparent material. The transparent material may allow the user to continuously inspect the tissue underneath, during application of the device and/or during child delivery.

The sheet of flexible material may have perforations or grooves. This helps potential fluids, such as bodily fluids to be removed from the area. This may be an advantage as fluids may compromise adhesion. This feature also protects the skin from any potential damage as prolonged exposure to liquids may damage the skin. Perforations may also enable the unskilled birth helper to inspect the skin through the perforations, which is useful in cases where the sheet is non-transparent.

The outer surface of the first part may be smooth with a low frictional resistance against human skin and hair, at least when wet. This may help the child slide over the device.

The material of the flexible sheet should be biocompatible and should not cause an allergic reaction. For this reason, latex or similar allergenic materials may be less preferred.

The sheet of flexible material may be cutable, allowing it to be cut to size. This is useful in cases where the area between the posterior part of the introitus vagina and anus is smaller than average. Another advantage of using cutable material is that an episiotomy may be performed without removing the device.

In one embodiment, the attachment element comprises an adhesive. This will help the device to remain in place during use.

The attachment element may be provided on the sheet of flexible material forming the second part or be integrated in the second part. The attachment element at the inner surface of the second part may be provided in that the inner surface of the second part is in itself adhering or in that an attachment element is provided on the inner surface. The attachment element is made of a material that adheres to surfaces of the female body, and preferably a material that adheres to surfaces of the body, which are covered with bodily fluids, such as vaginal secretion.

A separate attachment element may comprise an adhesive, such as a pressure-sensitive adhesive or glue, e.g. tissue specific glue. It may be advantageous that the attachment element is able to adhere to places where hair appears to ensure good adhesion.

In one embodiment, only the inner surface of the second part comprises an attachment element.

The attachment element may be covered by a cover in a state of delivery. This may prevent the device from unintentionally attaching to other items, such as other perineum damage-reducing devices.

The cover may comprise several separate elements or be divided into two or more sections, for example by perforations, such that sections of the attachment element may be uncovered independently. This will, for example, allow the middle section of a second part, which comprises a middle section and two side section as described above, to be uncovered and attached to the female body, while the side section remain covered. This will allow the application of the device, while some part of the attachment element remains covered, thus reducing the risk of unintentional attachment. Once the device has been positioned and attached at the middle section, the cover on the side sections can be removed and the side sections attached to the female body. Other application sequences are also possible.

In an embodiment, the device has a thickness of 0.1 mm to 10 mm, more preferably 0.5 mm to 5 mm. These thicknesses provide a balance between material consumption, ease of handling, and functionality. The thickness of the device may be selected based on functionality and the acceptance by the user, i.e. the device may from a technical perspective work at thicknesses down to 0.1 mm, but may be thicker to accommodate the user's perception and acceptance of it.

In an embodiment, the second part is thicker than the first part.

In one embodiment, the first part is thicker than the second part.

The thickness of the first part and/or of the second part may vary. For example, the second part may be thicker or thinner in the middle section compared to the side sections to provide desired physical properties, or the edges of the second part may be thinner than the rest to facilitate attachment or removal. As another example the edges of the first part may be thinner than a central section to make them more pliable than the central section, thereby potentially facilitating insertion or giving the first part a shape matching the shape of the vaginal wall.

A second aspect of the invention relates to a method for reducing damage during vaginal child delivery. The method includes providing a perineum damage-reducing device comprising a first part and a second part, where a sheet of flexible material defines an inner surface and an outer surface opposite to said inner surface, said inner surface extending over the first part and the second part, and where at least one reinforcing element extends over both the first part and the second part and stabilizes said sheet. The method further includes arranging the perineum damage-reducing device with the first part inserted into a female body via introitus vagina at a posterior part of introitus vagina and with the second part extending outside the female body away from the posterior part of introitus vagina towards anus, and such that the inner surface is in contact with surfaces of the female body, and attaching the second part to a surface between the posterior part of introitus vagina and anus of the female body using at least one attachment element at the inner surface.

Embodiments and advantages described with reference to one aspect of the invention also applies to the other unless otherwise stated. As an example, the possible removal of sections of a cover at different times during the application of a perineum damage-reducing device described above with reference to the first aspect also applies to the method.

### BRIEF DESCRIPTION OF DRAWINGS

In the following description, embodiments of the invention will be described with reference to the schematic drawing, in which:

FIG. 1 shows a first embodiment of a perineum damage-reducing device in a perspective view,

FIG. 2 is a front view of the device according to the first embodiment,

FIG. 3 is a side view of the device according to the first embodiment,

FIG. 4 is a cross-sectional view of a female body and a perineum damage-reducing device in use during vaginal child delivery,

FIG. 5 is a front view showing external female genital organs during vaginal child delivery, and a perineum damage-reducing device during use,

FIG. 6 is a perspective view of a second embodiment of the perineum damage-reducing device,

FIG. 7 is a perspective view of a third embodiment of the perineum damage-reducing device,

FIG. 8 is a perspective view of a fourth embodiment of the perineum damage-reducing device,

FIG. 9 is a perspective view of a fifth embodiment of the perineum damage-reducing device,

FIG. 10 is a perspective view of a sixth embodiment of the perineum damage-reducing device,

FIG. 11 is a front view showing external female genital organs during vaginal child delivery, and a perineum damage-reducing device during use,

FIG. 12 is a side view of the device according to the sixth embodiment of the perineum damage-reducing device,

FIG. 13 is a side view of a perineum damage-reducing device,

FIG. 14 is a cross-sectional view of a female body and a perineum damage-reducing device in use during vaginal child delivery,

FIG. 15 is a cross-sectional view of a female body and a perineum damage-reducing device in use during vaginal child delivery,

FIG. 16 is a cross-sectional view of a female body and a perineum damage-reducing device in use during vaginal child delivery,

FIG. 17 is a perspective view of a seventh embodiment of a perineum damage-reducing device,

FIG. 18 is a perspective view of an eighth embodiment of the perineum damage-reducing device,

FIG. 19 is a side view of the device according to the eighth embodiment of the perineum damage-reducing device,

FIG. 20 is a front view of a perineum damage-reducing device,

FIG. 21 is a front view of a perineum damage-reducing device,

FIG. 22 is a perspective view of a nineth embodiment of the perineum damage-reducing device,

FIG. 23 is a front view showing external female genital organs during vaginal child delivery, and the device according to the nineth embodiment during use,

FIG. 24 is a perspective view of a tenth embodiment of the perineum damage-reducing device, and

FIG. 25 is a front view showing external female genital organs during vaginal child delivery, and the device according to the tenth embodiment during use.

### DETAILED DESCRIPTION

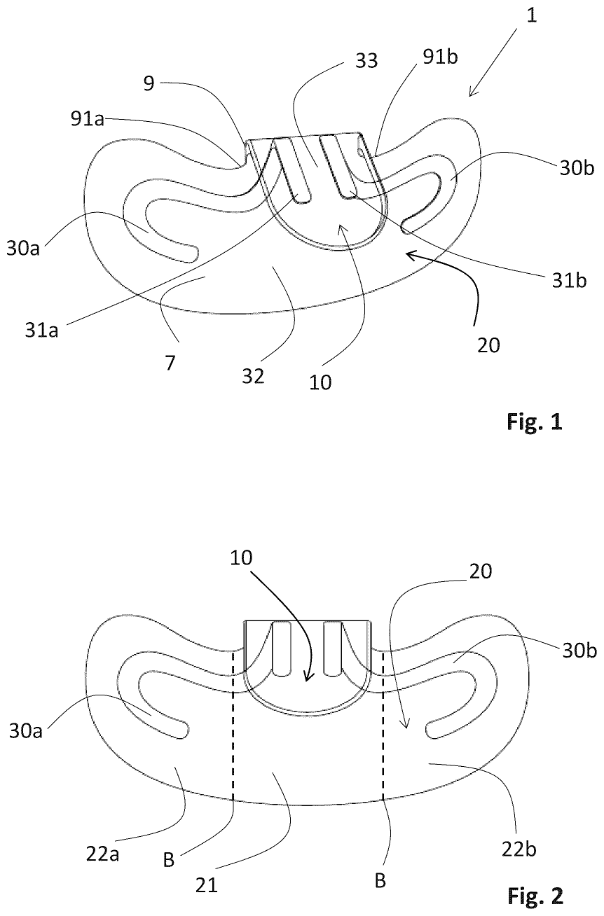

FIG. 1 and FIG. 2 are transparent illustrations of a first embodiment of a perineum damage-reducing device 1 having a first part 10 and a second part 20. An inner surface 32 extends over the first part 10 and the second part 20, and an outer surface 33 is opposite to said inner surface. Here, the inner surface 32 of the second part 20 and outer surface 33 of the first part 10 face the viewer.

The inner surface 32, the outer surface 33, and the overall shape of the device is defined by a sheet 7 of flexible material, which may for example be made from thermoplastic elastomers. Here, the embodiment is shown as see-through, such that reinforcing elements 30a, 30b inside the device are visible, however this may not be the case in other embodiments.

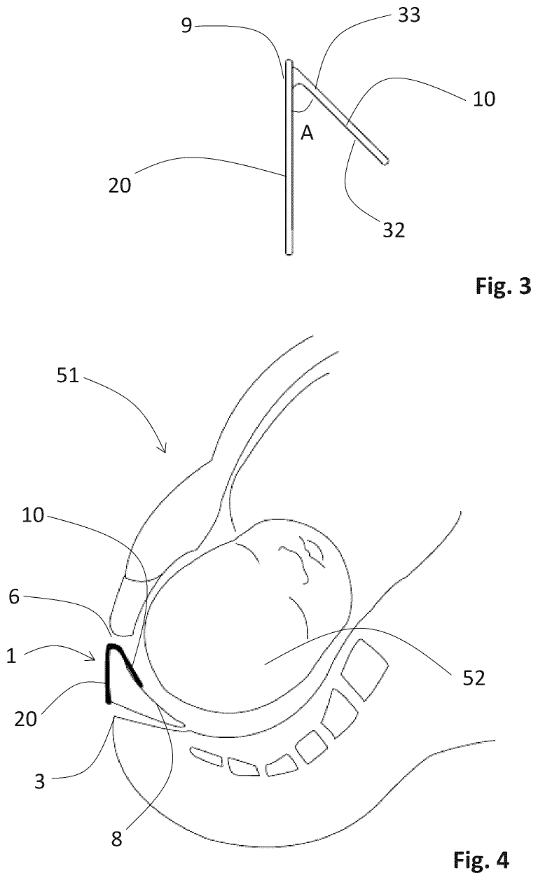

The device 1 is shown in a state where the first part 10 and the second part 20 meet at a bend 9 and extend at an angle A in relation to each other as is better seen in FIG. 3, which also shows that the second part 20 may be longer than the first part 10.

In FIG. 3, the angle A is approximately 45 degrees, but other angle sizes may be advantageous depending for example on the materials used for the device 1, the configurations of the first and second parts 10, 20, and the anatomy of the female body 51 on which the device is to be used.

The two reinforcing elements 30a, 30b extend from the first part 10 into a middle section 21 of the second part 20 and from there outwardly towards side sections 22a, 22b of the second part 20. The reinforcing elements 30a, 30b then extend back towards the middle section 21. This curved shape of the reinforcing elements 30a, 30b provides a large contact area between the sheet 7 and the reinforcing elements 30a, 30b, which provides stability to the device.

The reinforcing elements 30a, 30b are parallel to each other in the first part 10 to reinforce a wide range of the first part 10. The reinforcing elements may also be interconnected (not shown) at first ends 31a, 31b thereby forming one reinforcing element.

In the drawing, the reinforcing elements 30a, 30b are consistently shown as being flat, however they may have a rounded cross-sectional shape.

The perineum damage-reducing device 1 in FIG. 1 and FIG. 2 comprises recesses 91a, 91b at the bend 9. This may be advantageous to match the shape of some female bodies.

The middle section 21 and side sections 22a, 22b may be visibly distinguishable, for instance by providing visual indications at the broken lines B in FIG. 2, or they may be indistinguishable.

FIG. 4 is a principle sketch depicting a perineum damage-reducing device 1 applied to a female body 51 before a child 52 passes the vaginal opening 6. The first part 10 is in contact with a posterior part of the vaginal wall 8, i.e. the part closest to the anus 3. The second part 20 is attached to the area between a posterior part of the vaginal opening 6 and anus 3 to protect this area when the child passes through the vaginal opening 6. The angle A between the first part 10 and the second part 20 ensures a good fit of the device 1. As the physiology differs from person to person, the angle A may be adaptable and may change during child delivery. Different devices with different angles and/or sizes of the first and second parts may also be provided to be chosen from depending on the individual female body 51.

In this embodiment, the first part 10 is longer than the first part 10 according to the first embodiment. A shorter first part 10 may for example be advantageous if the device is to be applied late in the vaginal child delivery. However, a longer first part 10 may provide a larger contact area between the first part 10 and the vaginal wall 8 and may therefore provide better contact between the female body 51 and the device 1. As the physiology varies from country to country, both embodiments are preferred. The first part 10 may have different shapes and sizes in different embodiments depending for example on the anatomy of the female body 51. In this embodiment, the first part 10 has a rounded shape, however it may also have a more edgy shape as shown for example in FIG. 10.

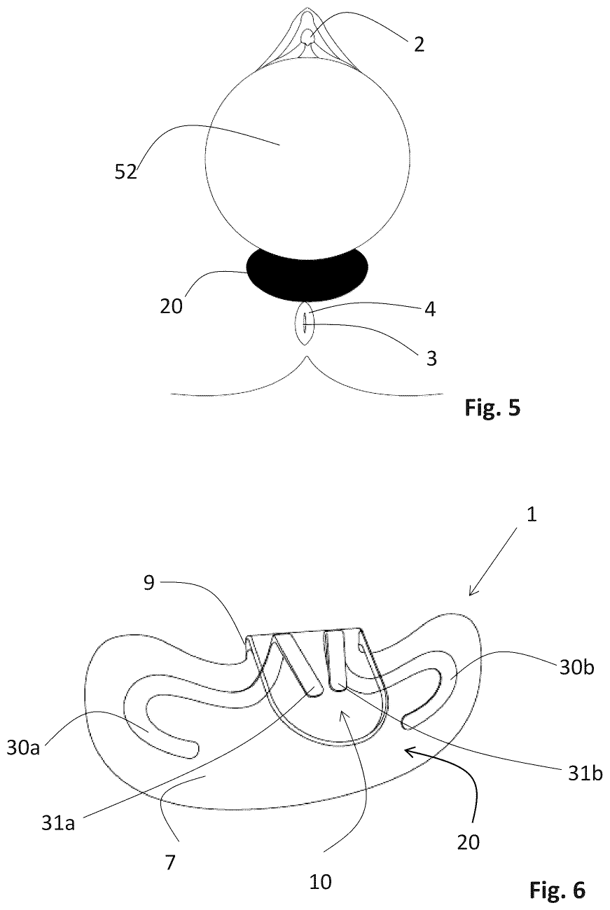

In FIG. 4 the first part 10 is depicted in the correct position, however this may not be the initial position. The first part 10 may have been repositioned to the correct position following the attachment of the second part 20, or following an expansion of the second part 20 as depicted in FIG. 5, where the head of the child 52 is passing the vaginal opening. The inner surface 32 of the first part 10 being smooth will contribute to allowing such a repositioning as will the reinforcing elements 30a, 30b.

FIG. 5 shows a principle sketch of external female genital organs, including urethral orifice 2, anus 3, anal sphincter 4, the child 52, and a perineum damage-reducing device 1 in use, where the second part 20 covers an area between the vaginal opening and the anus 3, also referred to as perineum. The child 52 is in the process of passing the vaginal opening 6, and as a result the length of the posterior part of the vaginal opening 6 may be doubled or tripled depending on the child 52 circumference and the presentation of the child's head. For this reason, the sheet may be made of elastic materials to ensure that the device does not break when being stretched. The reinforcing elements 30a, 30b provide additional stability to the device.

Ruptures often start at the posterior part of the vaginal opening 6, which is why protection of this area is important. The second part 20 adheres to at least a part of perineum to provide good protection to this area throughout the vaginal child delivery. The first part 10 is not visible in FIG. 5, as it is located in the vagina as shown in FIG. 4. The second part 20 is wider than the first part 10 so that the device may fit into the vaginal opening while still covering a large area of the perineum and adjacent areas.

In FIG. 5 the second part 20 has a size matching the distance between the vaginal opening 6 and the anus 3. If, however, this distance is smaller than average, the device may be cut such that the first part 10 and the second part 20 are equally long or such that the second part 20 is shorter than the first part 10. It is also possible to cut a local recess at the middle section of the second part. A cutting indication (not shown) may be provided to ensure that the cut does not affect the reinforcing elements.

In the following text, the same reference numbers are used for the parts that share the same function. Only differences will be described.

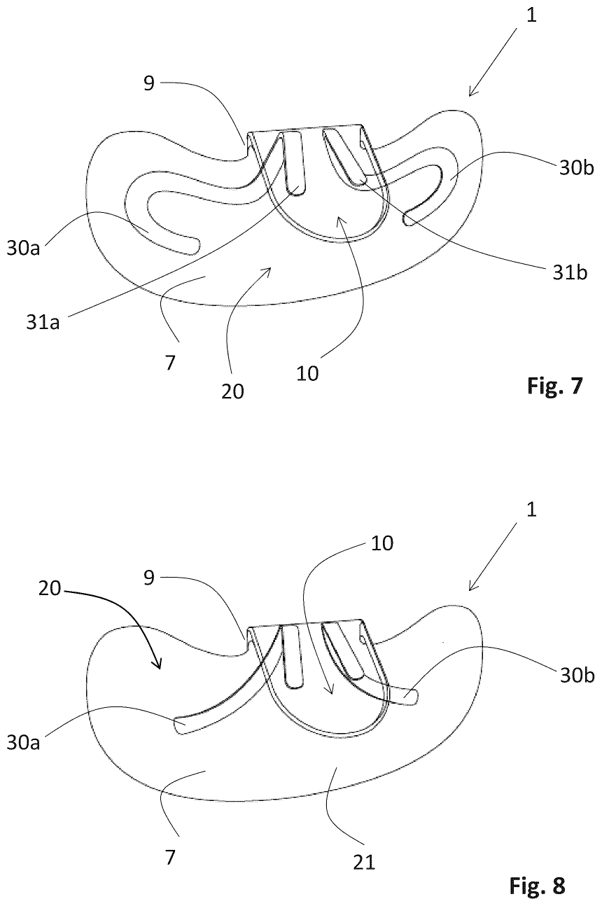

FIG. 6 and FIG. 7 show embodiments, which are similar to the one in FIG. 1, but where in FIG. 6 the first ends 31a, 31b of the reinforcing elements 30a, 30b extend towards each other in the first part 10 and in FIG. 7 the first ends 31a, 31b of the reinforcing elements 30a, 30b extend away from each other in the first part 10. The embodiment in FIG. 6 provides a particularly strong reinforcement at the center of the first part, whereas the embodiment in FIG. 7 may ensure that sides of the first part 10 do not collapse.

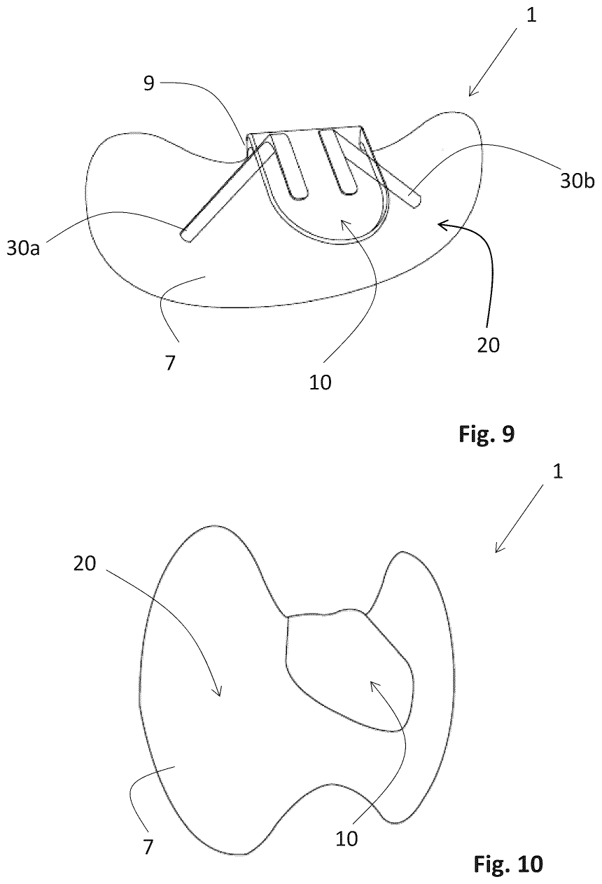

FIG. 8 and FIG. 9 also show embodiments, which are similar to the one in FIG. 1, but here the reinforcing elements 30a, 30b do not extend back towards the middle section 21. In these embodiments, the reinforcing elements 30a, 30b are shorter than in the previously described embodiment and has a smaller contact area with the sheet 7. This may be an advantage if the device 1 needs to be cut to suit dimensions of a small female body.

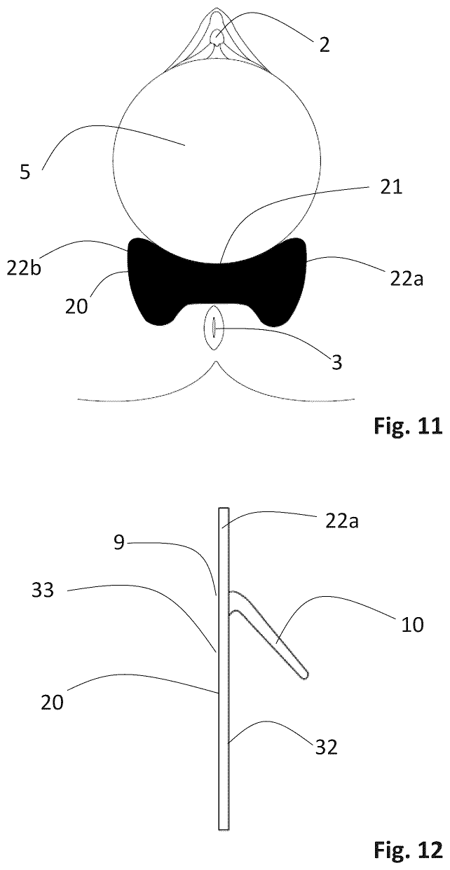

A further embodiment is shown in FIG. 10. Here, the sheet 7 has a butterfly shape to cover an area adjacent to the anus 3 and extending up along sides of the vaginal opening. This may result in a greater protection of the perineum, as the second part 20 covers a greater part of the perineum without covering anus, which is also shown in FIG. 11. Thus, forces are dispersed over a bigger area, thereby reducing the risk of damage to the perineum. Complete or partial attachment of the second part 20 to anus 3 may be uncomfortable to the female, and it may compromise attachment due to another tissue type and bodily fluids, which is why this should be avoided. In this embodiment, reinforcing elements are not visible, but are present inside the sheet 7.

FIG. 12 shows the embodiment in FIG. 11 in a side view, where the side sections 22a, 22b extend beyond the bend 9.

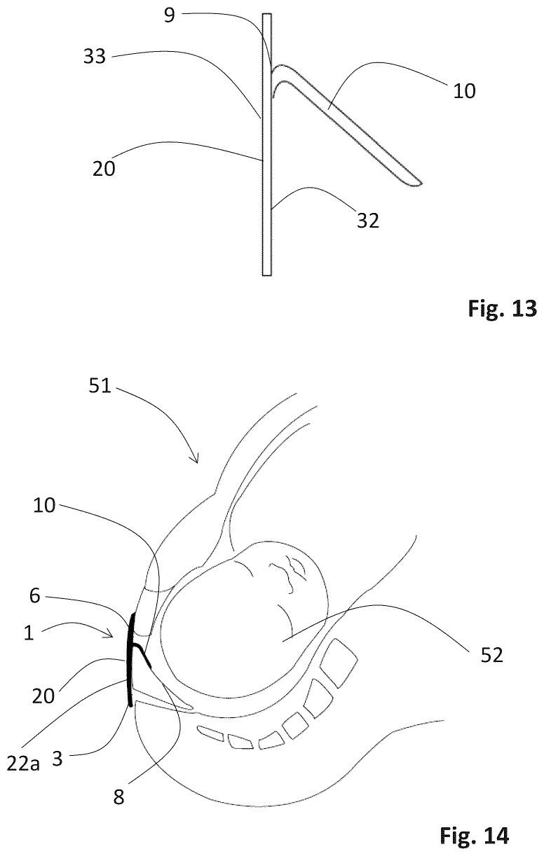

In the embodiment in FIG. 13, the side sections 22a, 22b extend beyond the bend to a lesser degree than the embodiment in FIG. 12, while the first part 10 is longer than in FIG. 12.

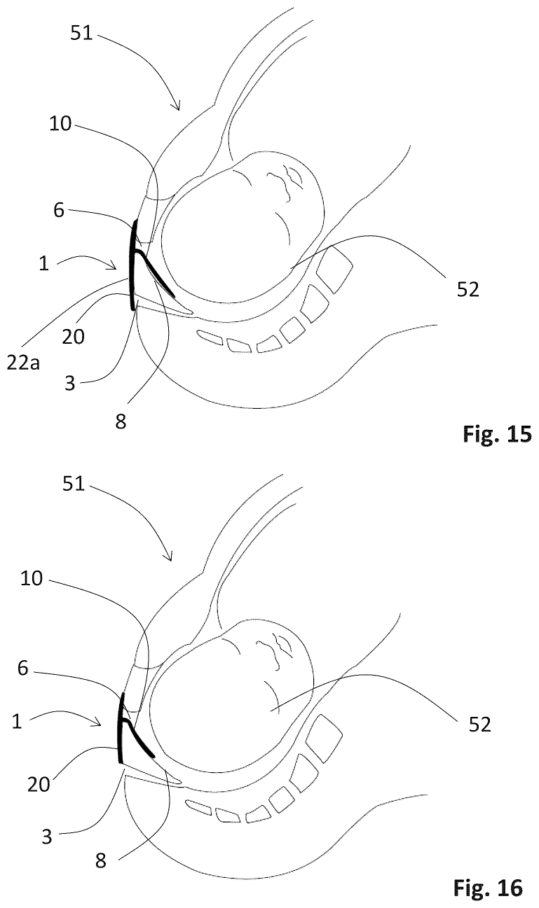

FIG. 14 is a principle sketch showing a perineum damage-reducing device 1 in use. In this embodiment, the first part 10 is shorter than the second part 20, and the side sections 22a, 22b extend past the anus 3. It is to be understood that the second part does not cover the anus, but extends on either side of it in the same way as shown in FIG. 11. The first part 10 may alternatively be longer as seen in FIG. 15. Embodiments, in which the device does not extend below anus are also possible as shown in FIG. 16, corresponding in principle to the situation in FIG. 5.

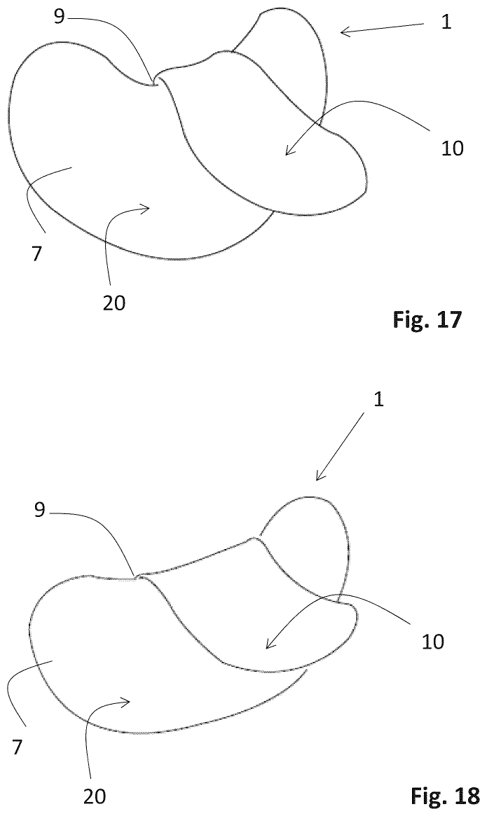

FIG. 17 depicts a perineum damage-reducing device 1, where the first part 10 is longer than in previously described embodiment and has a curvature in the state of delivery. The first part 10 is thicker at the tip and in the sides compared to the middle section. This may facilitate insertion in the vaginal opening.

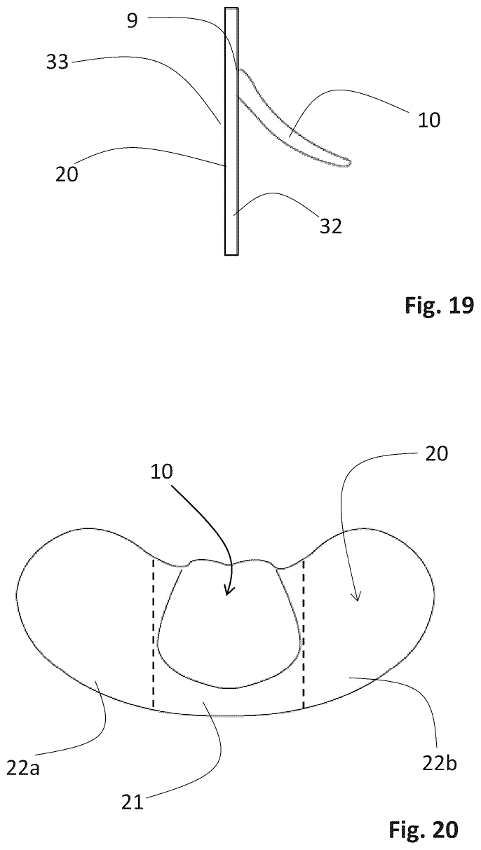

FIG. 18 and FIG. 19 show a further embodiment, in which the first part 10 has a curvature as in FIG. 17, but is slightly shorter, and where the second part 20 is also smaller than in FIG. 17. This embodiment may for example be advantageous if the female body is smaller than average. As opposed to the embodiment in FIGS. 1 and 2, the embodiment in FIG. 18 has no recesses at the bend 9. This may provide a strength advantage.

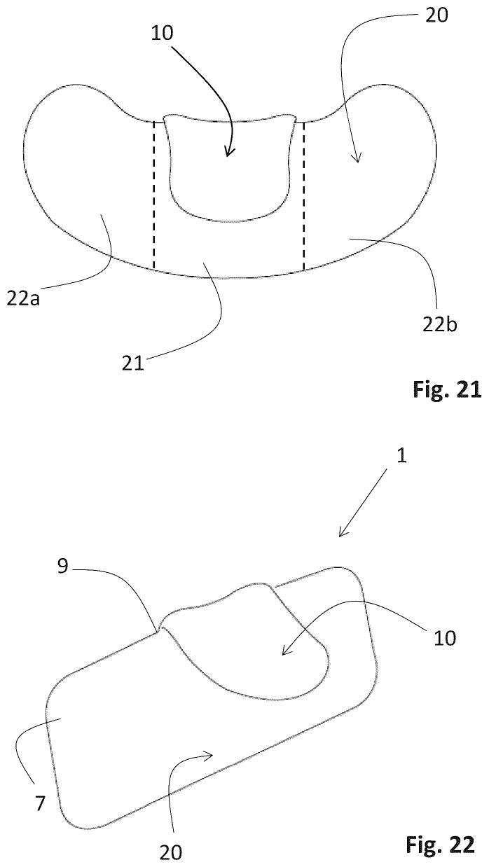

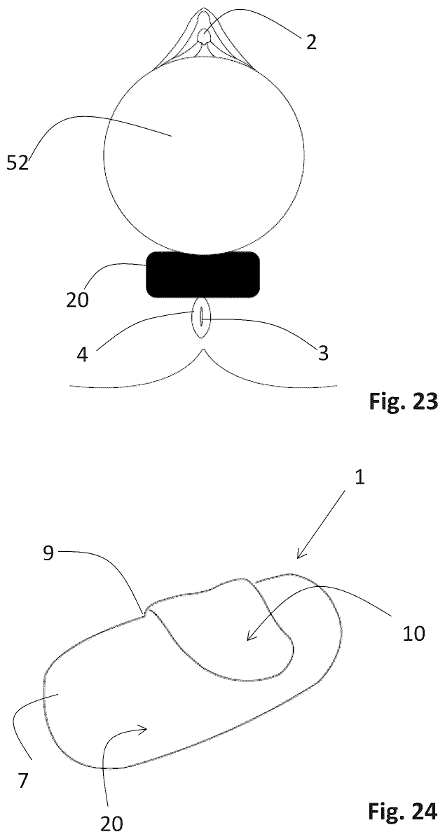

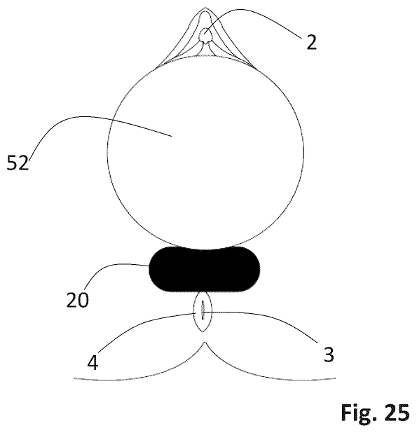

Further embodiments showing possible variations of sizes and shapes of the first part 10 and the second part 20 are shown in FIG. 20-25. It is to be understood that these are just examples and that further variations and combinations are within the scope of the invention.

### LIST OF REFERENCE NUMERALS

1 Device
2 Urethral orifice
3 Anus
4 Anal sphincter
51 Female body
52 Child
6 Vaginal opening
7 Sheet of flexible material
8 Vaginal wall
9 Bend
91a Recess
91b Recess
10 First part
20 Second part
21 Middle section
22a Side sections
22b Side sections
30a Reinforcing element
30b Reinforcing element
31a First end of reinforcing element 30a
31b First end of reinforcing element 30b
32 Inner surface
33 Outer surface

1. A perineum damage-reducing device for reducing damage during vaginal child delivery, comprising:
a first part configured to be inserted into a female body via introitus vagina at a posterior part of introitus vagina;
a second part configured to extend outside the female body away from the posterior part of introitus vagina towards anus;
an inner surface extending over the first part and the second part and being configured to be in contact with surfaces of the female body;
an outer surface opposite to said inner surface;
at least one reinforcing elements for stabilizing said sheet;
wherein:
said inner surface and said outer surface are formed by a sheet of flexible material;
the at least one reinforcing element extends over both said first and said second part; and
the second part comprises at least one attachment element at the inner surface configured for attachment to a surface of the female body.

2. The perineum damage-reducing device according to claim 1, wherein the inner surface of said first part comprises a smooth surface.

3. The perineum damage-reducing device according to claim 1, wherein the second part comprises two side sections and a middle section, and wherein the at least one reinforcing element extends through the middle section and outwardly towards edges of said side sections.

4. The perineum damage-reducing device according to claim 3, wherein the at least one reinforcing element further extends inwardly back towards the middle section of the second part.

5. The perineum damage-reducing device according to claim 1, wherein said first part and said second part meet at a bend and extend at an angle in relation to each other, said angle being in the range of 20-70 degrees.

6. The perineum damage-reducing device according to claim 5, wherein, in a state of delivery the device is pre-formed with the bend between the first part and the second part.

7. The perineum damage-reducing device according to claim 5, wherein the sheet of flexible material has a thickening at the bend.

8. The perineum damage-reducing device according to claim 1, wherein the at least one reinforcing element is elastic, pulling the device towards a state of delivery.

9. The perineum damage-reducing device according to claim 1, wherein the reinforcing element has a higher stiffness than the sheet.

10. The perineum damage-reducing device according to claim 1, wherein the reinforcing element is embedded in the sheet of flexible material between said inner surface and said outer surface.

11. The perineum damage-reducing device according to claim 1, wherein the attachment element comprises an adhesive.

12. The perineum damage-reducing device according to claim 1, wherein the device has a thickness of 0.1 mm to 10 mm.

13. A method for reducing damage during vaginal child delivery, comprising:
providing a perineum damage-reducing device comprising a first part and a second part, where a sheet of flexible material defines an inner surface and an outer surface opposite to said inner surface, said inner surface extending over the first part and the second part, and where at least one reinforcing element extends over both the first part and the second part and stabilizes said sheet;
arranging the perineum damage-reducing device with the first part inserted into a female body via introitus vagina at a posterior part of introitus vagina and with the second part extending outside the female body away from the posterior part of introitus vagina towards anus, wherein the inner surface is in contact with surfaces of the female body and
attaching the second part to a surface between the posterior part of introitus vagina and anus of the female body using at least one attachment element at the inner surface.

14. A method according to claim 13, where the perineum damage-reducing device is bent before being arranged on the female body so that the first part and said second part extend at an angle in relation to each other.

15. The perineum damage-reducing device according to claim 1, wherein said first part and said second part meet at a bend and extend at an angle in relation to each other, said angle being in the range of 30-40 degrees.

16. The perineum damage-reducing device according to claim 6, wherein the sheet of flexible material has a thickening at the bend.

17. The perineum damage-reducing device according to claim 1, wherein the device has a thickness of 0.5 mm to 5 mm.
